(19) United States
(12) Patent Application Publication
(10) Pub. No.: US 2022/0073898 A1
(43) Pub. Date: Mar. 10, 2022

(54) Tandem Protein Expression

(71) Applicant: Novozymes A/S, Bagsvaerd (DK)

(72) Inventors: Richard Jan Steven Baerends, Bagsværd (DK); Carsten Lillelund Olsen, Bagsvaerd (DK); Josè Arnau, Hellerup (DK)

(73) Assignee: Novozymes A/S, Bagsvaerd (DK)

(21) Appl. No.: 17/416,752

(22) PCT Filed: Dec. 17, 2019
(86) PCT No.: PCT/EP2019/085553

§ 371 Date: Jun. 21, 2021

(30) Foreign Application Priority Data
Dec. 21, 2018 (EP) 18215558.0

(51) Int. Cl. C12N 9/96; C12N 15/62; C12N 15/80

(52) U.S. Cl. CPC C12N 9/96; C12N 15/625; C07K 2319/02; C07K 2319/50; C12N 15/80

(57) ABSTRACT

The present invention provides means and methods for improving polypeptide expression yield by increasing the number of polypeptides that can be transported into the endoplasmic reticulum (ER) per translocation event.

### REFERENCE TO SEQUENCE LISTING

This application contains a Sequence Listing in computer readable form. The computer readable form is incorporated herein by reference.

### FIELD OF THE INVENTION

The present invention provides means and methods for improving polypeptide expression yield by increasing the number of polypeptides that can be transported into the endoplasmic reticulum (ER) per translocation event.

### BACKGROUND OF THE INVENTION

Product development in industrial biotechnology includes a continuous challenge to increase enzyme yields at large scale to reduce costs. Two major approaches have been used for this purpose in the last decades. The first one is based on classical mutagenesis and screening. Here, the specific genetic modification is not pre-defined and the main requirement is a screening assay that is sensitive to detect relatively discrete increments in yield. High throughput screening enables large numbers of mutants to be screened in search for the desired phenotype, i.e., higher enzyme yields. The second approach includes numerous strategies ranging from the use of stronger promoters and multicopy strains to ensure high expression of the gene of interest to the use of codon optimized gene sequences to aid translation. However, high level production of a protein may trigger several bottlenecks in the cellular machinery for secretion of the enzyme of interest into the medium.

For secreted enzymes whose amino acid sequence includes a signal peptide (SP), translation is followed by cleavage of the SP by a signal peptidase and translocation of the maturing protein into the endoplasmic reticulum (ER; Voss et al., 2013; Aviram and Schuldiner 2017). To secrete a protein through the ER, the signal recognition particle (SRP) recognizes the SP in a highly conserved manner. The SRP associates with the ribosome and through a hydrophobic cleft recognizes secretory proteins with hydrophobic motifs as they are being translated and binds to the SRP receptor (SR) present in the ER membrane in eukaryotes (Aviram and Schuldiner 2017). Thus, a limiting step in the secretion of a protein might be at the translocation of the nascent polypeptide from the cytosol into the ER. Since this process is very energy demanding, there is an upper capacity limit as to the number of translocations per time unit per cell.

Means and methods for overcoming the upper translocation limit would constitute an entirely new approach to yield optimization and would thus be highly desirable within industrial biotechnology,

### SUMMARY OF THE INVENTION

The present invention provides means and methods for improving polypeptide expression yield by increasing the number of polypeptides that can be transferred into the ER while still using one signal peptide per translocation-dependent event, thereby increasing the number of polypeptide units that can be secreted per translocation event.

In a first aspect, the present invention relates to a nucleic acid construct comprising a heterologous promoter operably linked to:

a) a polynucleotide encoding a signal peptide; and

b) at least two polynucleotides encoding one or more polypeptide of interest;

wherein the at least two polynucleotides encoding one or more polypeptide of interest are each separated by a linker polynucleotide encoding a linker polypeptide comprising a proteolytic cleavage site; and

wherein the signal peptide, the one or more polypeptide of interest and the linker poly-peptide(s) comprising a proteolytic cleavage site are encoded in frame as a single polypeptide.

In a second aspect, the present invention relates to an expression vector comprising a nucleic acid construct of the first aspect.

In a third aspect, the present invention relates to a fungal host cell comprising a nucleic acid construct of the first aspect and/or an expression vector of the second aspect.

In a fourth aspect, the present invention relates to a method for producing one or more polypeptide of interest, the method comprising:

a) providing a fungal host cell of the third aspect:

b) cultivating said host cell under conditions conducive for expression of the one or more polypeptide of interest; and optionally

c) recovering the one or more polypeptide of interest.

### BRIEF DESCRIPTION OF DRAWINGS

FIG. 1 shows schematically the lipVR expression cassette. Shortly, one or more “units” of the lipVR coding sequence (using different codon optimized version of the gene, lipVR, lipVR*, etc.) were cloned to make genetic constructions where lipVR is transcribed as a singlet, tandem or more. SP: native signal peptide from the lipVR gene or the cutinase prepro encoding sequence; CP: cutinase pro encoding sequence; KexB: KexB cleavage site (“KR”). In all cases, a single transcript contains all the “lipVR units”.

FIG. 2 shows a map of plasmid pAT652 for expression and secretion of the LipVR lipase in A. oryzae (single gene construct).

FIG. 3 shows a map of plasmid pAT1509 for expression and secretion of the LipVR lipase in A. oryzae (tandem gene construct).

FIG. 4 (left) shows LipVR production as demonstrated by SDS PAGE for comparison of singlet vs. tandem production of LipVR by A. oryzae strains AT969 (lane 1: 18 copies of single expression plasmid pAT652) and AT1684 (lane 2: 9 copies of pAT1509 (lipVR tandem)) at the end of fermentation (day 7). FIG. 4 (right) shows lipolytic activity (depicted as arbitrary units, A.U.) at the end of fermentation of strain AT969 and AT1684.

FIG. 5 shows the degree of correct processing to yield intact LipVR molecules produced during fed-batch fermentation of A. oryzae strain AT1684 containing the lipVR-tandem cassette. Samples (supernatant) were taken at the end of fermentation at 30° C. or 34° C. and at pH 6.5 or 7.4 as indicated and were analyzed by mass spectrometry. The level of tandem processing is shown as percentage of full length LipVR molecules (black boxes) and the aggregated percentages of differently but non-fully processed LipVR molecules (grey boxes).

### DEFINITIONS

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor mRNA that is processed through a series of steps, including RNA splicing, before appearing as mature mRNA.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Heterologous promoter: The term “heterologous promoter” means a promoter that is foreign (i.e., from a different gene) to the polynucleotide to which it is operably linked.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 289 of SEQ ID NO: 5 (corresponding to amino acids 1 to 289 of SEQ ID NO: 6). Amino acids −20 to −1 of SEQ ID NO: 5 are a signal peptide. It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

Mature polypeptide coding sequence: The term “mature polypeptide coding sequence” means a polynucleotide that encodes a mature polypeptide having lipolytic activity as determined in WO 2018/150021. In one aspect, the mature polypeptide coding sequence is nucleotides 61 to 1056 of SEQ ID NO: 3 or the cDNA sequence thereof (corresponding to nucleotides 61 to 927 of SEQ ID NO: 4) and nucleotides 1 to 60 of SEQ ID NO: 3 (corresponding to nucleotides 1 to 60 of SEQ ID NO: 4) encode a signal peptide.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Secreted: The term “secreted” means that the one or more polypeptide of interest is processed and released into an extracellular environment, such as a growth medium. In one embodiment, the one or more polypeptide of interest is processed and released via the conventional secretion pathway comprising the endoplasmatic reticulum, Golgi apparatus, and secretory vesicles. In another embodiment, the one or more polypeptide of interest is processed and released via the unconventional secretion pathway (Rabouille, Trends in Cell Biology 2017, vol. 27, pp. 230-240; Kim et al., Journal of Cell Science 2018, vol. 131, jcs213686).

Sequence identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the-nobrief option) is used as the percent identity and is calculated as follows:

(Identical Deoxyribonucleotides×100)/(Length of Alignment−Total Number of Gaps in Alignment)

Variant: The term “variant” means a polypeptide comprising an alteration, i.e., a substitution, insertion, and/or deletion, at one or more (e.g., several) positions compared to the corresponding native polypeptide. A substitution means replacement of the amino acid occupying a position with a different amino acid; a deletion means removal of the amino acid occupying a position; and an insertion means adding an amino acid adjacent to and immediately following the amino acid occupying a position.

### DETAILED DESCRIPTION OF THE INVENTION

The present invention provides means and methods for improving polypeptide expression yield by increasing the number of polypeptides that can be transported into the ER while still using only one SP per translocation event, thereby increasing the number of polypeptide units that can be secreted per translocation event.

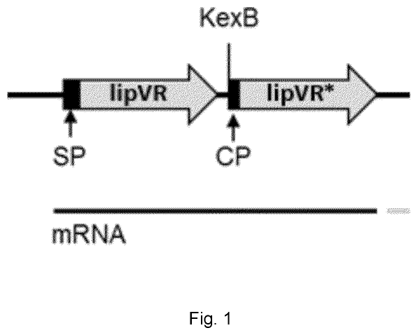

The inventive concept is based on the design of nucleic acid constructs comprising a heterologous promoter operably linked to a polynucleotide encoding a signal peptide and at least two polynucleotides encoding one or more polypeptide of interest. Each of the polynucleotides encoding a polypeptide of interest are separated by a linker polynucleotide that encodes a linker polypeptide comprising a proteolytic cleavage site that is recognized by a suitable protease (FIG. 1). The signal peptide, the one or more polypeptide of interest, and the linker polypeptide(s) are encoded in frame to allow translation as a single polypeptide. The term “encoded in frame” refers to the mature mRNA that is obtained after RNA splicing and removal of any introns present in the precursor mRNA.

The nucleic acid constructs of the invention may be transformed into suitable host cells, and fermentation of such host cells under suitable conditions will allow transcription of the polynucleotides followed by ribosomal translation of the resulting mRNA. The resulting polypeptide containing one signal peptide and one or more polypeptide of interest each separated by a linker polypeptide will subsequently be targeted into the secretory pathway as a single unit in one translocation event. Following proteolytic processing by a protease that recognizes the proteolytic cleavage site comprised by the linker polypeptide, the one or more polypeptide of interest is liberated and secreted into the extracellular medium.

As shown in the Examples disclosed herein, use of nucleic acid constructs of the invention in the construction of fungal host cells results in increased yield of the polypeptide of interest as well as uniform processing of the individual copies of the polypeptide of interest. The latter is especially important for functional polypeptides such as enzymes, since integrity of the primary structure is important for folding and correct formation of the tertiary structure, which is important for activity.

Thus, in a first aspect, the present invention relates to nucleic acid construct comprising a heterologous promoter operably linked to:

a) a polynucleotide encoding a signal peptide; and

b) at least two polynucleotides encoding one or more polypeptide of interest;

wherein the at least two polynucleotides encoding one or more polypeptide of interest are each separated by a linker polynucleotide encoding a linker polypeptide comprising a proteolytic cleavage site; and

wherein the signal peptide, the one or more polypeptide of interest and the linker poly-peptide(s) comprising a proteolytic cleavage site are encoded in frame as a single polypeptide.

Preferably, the nucleic acid constructs of the invention comprise at least two, e.g., 2, 3, 4, 5, 6, 7, 8, 9, 10, 11, 12, 13, 14, 15, 20, 25, 30, 35, 40, or more, polynucleotides encoding a one or more, e.g., 1, 2, 3, 4, 5, 6, 7, 8, 9, 10, 11, 12, 13, 14, 15, 20, 25, 30, 35, 40, or more, polypeptide of interest.

The signal peptide may be any signal peptide that is suitable for targeting the one or more polypeptide of interest to the secretory pathway of a host cell, preferably a fungal host cell. Preferably, the signal peptide is a heterologous signal peptide.

In a preferred embodiment, the signal peptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 2 preferably the signal peptide comprises or consists of SEQ ID NO: 2.

In a preferred embodiment, the polynucleotide encoding the signal peptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 1; preferably the polynucleotide encoding the signal peptide comprises or consists of SEQ ID NO: 1.

The one or more polypeptide of interest may be any polypeptide. Preferably, the polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the enzyme is a lipase.

In a preferred embodiment, the one or more polypeptide of interest has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to the mature polypeptide of SEQ ID NO: 5; preferably the one or more polypeptide of interest comprises or consists of the mature polypeptide of SEQ ID NO: 5.

In a preferred embodiment, the at least two polynucleotides encoding one or more polypeptide of interest have a sequence identity of, independently, at least 80%, e.g., at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO: 4; preferably the at least two polynucleotides encoding one or more polypeptide of interest comprise or consist of the mature polypeptide coding sequence of SEQ ID NO: 4; also preferably, the at least two polynucleotides encoding one or more polypeptide are different, i.e., not identical.

In another preferred embodiment, the nucleic acid constructs of the invention comprise at least two polynucleotides encoding at least two polypeptides of interest having the same amino acid sequence, and the at least two polypeptides of interest are secreted as separate polypeptides having the same amino acid sequence. Preferably, the at least two polypeptides of interest have a sequence identity of at least 80%, e.g., at least 85%, at least 90%, at least 91%, at least 92%, at least 93%, at least 94%, at least 95%, at least 96%, at least 97%, at least 98%, at least 99%, or 100%, to the mature polypeptide of SEQ ID NO: 5; preferably the at least two polypeptides of interest comprise or consist of the mature polypeptide of SEQ ID NO: 5. Preferably, the at least two polynucleotides encoding the at least two polypeptides of interest have a sequence identity of, independently, at least 80%, e.g., at least 85%, at least 90%, at least 91%, at least 92%, at least 93%, at least 94%, at least 95%, at least 96%, at least 97%, at least 98%, at least 99%, or 100%, to the mature polypeptide coding sequence of SEQ ID NO: 4; preferably the at least two polynucleotides encoding the at least two polypeptides of interest comprise or consist of the mature polypeptide coding sequence of SEQ ID NO: 4; also preferably, the at least two polynucleotides encoding the at least two polypeptides are different, i.e., not identical.

The linker polynucleotide should be chosen so that the encoded linker polypeptide is of sufficient length and flexibility to allow the proteolytic cleavage site to be accessed by a suitable protease that recognizes the proteolytic cleavage site. Preferably, the linker polypeptide comprise at least 10 amino acids, e.g., at least 11 amino acids, at least 12 amino acids, at least 13 amino acids, at least 14 amino acids, at least 15 amino acids, at least 16 amino acids, at least 17 amino acids, at least 18 amino acids, at least 19 amino acids, at least 20 amino acids, at least 25 amino acids, at least 30 amino acids, at least 35 amino acids, at least 40 amino acids, at least 45 amino acids, at least 45 amino acids, at least 50 amino acids, at least 55 amino acids, at least 60 amino acids, at least 65 amino acids, at least 70 amino acids, at least 75 amino acids, at least 80 amino acids, at least 85 amino acids, at least 90 amino acids, at least 95 amino acids, at least 100 amino acids, or more.

The linker polypeptide comprises a proteolytic cleavage site that is recognized by a suitable protease, preferably a KexB protease. Preferably, the proteolytic cleavage site is a dibasic amino acid motif such as a Lys-Arg or an Arg-Arg motif. Preferably, the proteolytic cleavage site is a KexB cleavage site.

The one or more polypeptide of interest is expressed as part of a single polypeptide, but should be liberated intracellularly prior to secretion of the individual polypeptides of interest into the extracellular medium. Thus, preferably, the proteolytic cleavage site encoded by the linker polypeptide is recognized by an intracellular protease. Preferably, proteolytic cleavage takes place intracellularly inside a suitable host cell comprising a nucleic acid construct of the invention. Alternatively, proteolytic cleavage takes place extracellularly during and/or after cultivation of a suitable host cell comprising a nucleic acid construct of the invention.

### Nucleic Acid Constructs

The present invention relates to nucleic acid constructs comprising polynucleotides encoding a signal peptide, one or more polypeptide of interest, and linker polypeptide(s) operably linked to one or more control sequences that direct the expression of the polynucleotides in a suitable host cell under conditions compatible with the control sequences.

The polynucleotides may be manipulated in a variety of ways to provide for their expression. Manipulation of a polynucleotide may be desirable or necessary depending on the nucleic acid construct, expression vector, and/or host cell into which the polynucleotide is being introduced. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The selected promoter should be a heterologous promoter that is foreign (i.e., from a different gene) to the polynucleotide(s) to which it is operably linked.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

In a preferred embodiment, the signal peptide has a sequence identity of at least 80%, e.g., at least 85%, at least 90%, at least 91%, at least 92%, at least 93%, at least 94%, at least 95%, at least 96%, at least 97%, at least 98%, at least 99%, or 100%, to SEQ ID NO: 2; preferably the signal peptide comprises or consists of SEQ ID NO: 2.

In a preferred embodiment, the signal peptide comprises or consists of SEQ ID NO: 2.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally in-active and can be converted to an active polypeptide by catalytic or autocatalytic cleavage of the propeptide from the propolypeptide. The propeptide coding sequence may be obtained from the genes for Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound.

In yeast, the ADH2 system or GAL1 system may be used.

In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used.

Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Polynucleotides

The present invention also relates to polynucleotides encoding a signal peptide, polypeptides encoding one or more polypeptide of interest, and linker polynucleotide(s) encoding linker polypeptide(s). In an embodiment, the polynucleotides have been isolated.

The techniques used to isolate or clone a polynucleotide are known in the art and include isolation from genomic DNA or cDNA, or a combination thereof. The cloning of the polynucleotides from genomic DNA can be effected, e.g., by using the well-known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus, or a related organism and thus, for example, may be an allelic or species variant of the polypeptide encoding region of the polynucleotide.

Modification of a polynucleotide encoding a signal peptide, one or more polypeptide of interest, or linker polypeptide may be necessary for synthesizing polypeptides substantially similar to these polypeptides. The term “substantially similar” to the polypeptide refers to non-naturally occurring forms of the polypeptide.

### Expression Vectors

In a second aspect, the present invention also relates to recombinant expression vectors comprising polynucleotides of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Suitable selectable markers for yeast host cells include, but are not limited to, ADE2, HIS3, LEU2, LYS2, MET3, TRP1, and URA3.

Suitable selectable markers for filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome. For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

In a third aspect, the present invention also relates to recombinant host cells comprising a nucleic acid construct or expression vector of the invention. A construct or vector comprising polynucleotides of the invention is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of host cell will to a large extent depend upon the polynucleotides encoding the one or more polypeptide of interest and their source.

The host cell may be any cell useful in the recombinant production of a polypeptide of interest, e.g., a eukaryotic cell, preferably a fungal cell.

“Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis sub-rufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

In a fourth aspect, the present invention also relates to methods of producing one or more polypeptide of interest, comprising:

a) providing a fungal host cell of the present invention;

b) cultivating the host cell under conditions conducive for production of the one or more polypeptide of interest; and optionally

c) recovering the one or more polypeptide of interest.

In one embodiment, the host cell is an Aspergillus cell. In a preferred embodiment, the host cell is an Aspergillus oryzae cell.

The fungal host cells are cultivated in a nutrient medium suitable for production of the polypeptide of interest using methods known in the art. For example, the cells may be cultivated by shake flask cultivation, or small-scale or large-scale fermentation (including continuous, batch, fed-batch, or solid-state fermentations) in laboratory or industrial fermentors in a suitable medium and under conditions allowing the polypeptide of interest to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide of interest is secreted into the nutrient medium, the polypeptide of interest can be recovered directly from the medium. If the polypeptide of interest is not secreted, it can be recovered from cell lysates.

The one or more polypeptide of interest may be detected using methods known in the art that are specific for the polypeptide. These detection methods include, but are not limited to, use of specific antibodies, formation of an enzyme product, or disappearance of an enzyme substrate. For example, an enzyme assay may be used to determine the activity of the polypeptide of interest.

The one or more polypeptide of interest may be recovered using methods known in the art. For example, the one or more polypeptide of interest may be recovered from the nutrient medium by conventional procedures including, but not limited to, collection, centrifugation, filtration, extraction, spray-drying, evaporation, or precipitation. In one aspect, a fermentation broth comprising the one or more polypeptide of interest is recovered.

The one or more polypeptide of interest may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the one or more polypeptide of interest is not recovered, but rather a host cell of the present invention expressing the one or more polypeptide of interest is used as a source of the polypeptide(s).

### Preferred Embodiments

1) A nucleic acid construct comprising a heterologous promoter operably linked to:

a) a polynucleotide encoding a signal peptide; and

b) at least two polynucleotides encoding one or more polypeptide of interest;

wherein the at least two polynucleotides encoding one or more polypeptide of interest are each separated by a linker polynucleotide encoding a linker polypeptide comprising a proteolytic cleavage site; and

wherein the signal peptide, the one or more polypeptide of interest and the linker poly-peptide(s) comprising a proteolytic cleavage site are encoded in frame as a single polypeptide.

2) The nucleic acid construct according to claim 1, wherein the at least two polynucleotides encode at least two polypeptides of interest having the same amino acid sequence, and wherein the at least two polypeptides of interest are secreted as separate polypeptides having the same amino acid sequence.
3) The nucleic acid construct according any of the preceding embodiments, wherein the signal peptide has a sequence identity of at least 80% to SEQ ID NO: 2; preferably the signal peptide comprises or consists of SEQ ID NO: 2.
4) The nucleic acid construct according to any of the preceding embodiments, wherein the polynucleotide encoding the signal peptide has a sequence identity of at least 80% to SEQ ID NO: 1; preferably the polynucleotide encoding the signal peptide comprises or consists of SEQ ID NO: 1.
5) The nucleic acid construct according to any of the preceding embodiments, wherein the one or polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the polypeptide of interest is a lipase.
6) The nucleic acid construct according to any of the preceding embodiments, wherein the one or more polypeptide of interest has a sequence identity of at least 80% to the mature polypeptide of SEQ ID NO: 5; preferably the one or more polypeptide of interest comprises or consists of the mature polypeptide of SEQ ID NO: 5.
7) The nucleic acid construct according to any of the preceding embodiments, wherein the at least two polynucleotides encoding one or more polypeptide of interest have a sequence identity of, independently, at least 80% to the mature polypeptide coding sequence of SEQ ID NO: 4; preferably the at least two polynucleotides encoding one or more polypeptide of interest comprise or consist of the mature polypeptide coding sequence of SEQ ID NO: 4.
8) The nucleic acid construct according to any of the preceding embodiments, wherein the linker polypeptide comprises at least 10 amino acids.
9) The nucleic acid construct according to any of the preceding embodiments, wherein the proteolytic cleavage site is a dibasic amino acid motif; preferably the proteolytic cleavage site is a Lys-Arg motif or an Arg-Arg motif.
10) The nucleic acid construct according to any of the preceding embodiments, wherein the proteolytic cleavage site is a KexB cleavage site.
11) An expression vector comprising a nucleic acid construct comprising a heterologous promoter operably linked to:

a) a polynucleotide encoding a signal peptide; and

b) at least two polynucleotides encoding one or more polypeptide of interest;

wherein the at least two polynucleotides encoding one or more polypeptide of interest are each separated by a linker polynucleotide encoding a linker polypeptide comprising a proteolytic cleavage site; and

wherein the signal peptide, the one or more polypeptide of interest and the linker polypeptide(s) comprising a proteolytic cleavage site are encoded in frame as a single polypeptide.

12) The expression vector according to claim 11, wherein the at least two polynucleotides encode at least two polypeptides of interest having the same amino acid sequence, and wherein the at least two polypeptides of interest are secreted as separate polypeptides having the same amino acid sequence.
13) The expression vector according to any of embodiments 11-12, wherein the signal peptide has a sequence identity of at least 80% to SEQ ID NO: 2; preferably the signal peptide comprises or consists of SEQ ID NO: 2.
14) The expression vector according to any of embodiments 11-3, wherein the polynucleotide encoding the signal peptide has a sequence identity of at least 80% to SEQ ID NO: 1; preferably the polynucleotide encoding the signal peptide comprises or consists of SEQ ID NO: 1.
15) The expression vector according to any of embodiments 11-14, wherein the one or polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the one or more polypeptide of interest is a lipase.
16) The expression vector according to any of embodiments 11-15, wherein the one or more polypeptide of interest has a sequence identity of at least 80% to the mature polypeptide of SEQ ID NO: 5; preferably the one or more polypeptide of interest comprises or consists of the mature polypeptide of SEQ ID NO: 5.
17) The expression vector according to any of embodiments 11-16, wherein the at least two polynucleotides encoding one or more polypeptide of interest have a sequence identity of, independently, at least 80% to the mature polypeptide coding sequence of SEQ ID NO: 4; preferably the at least two polynucleotides encoding one or more polypeptide of interest comprise or consist of the mature polypeptide coding sequence of SEQ ID NO: 4.
18) The expression vector according to any of embodiments 11-17, wherein the linker polypeptide comprises at least 10 amino acids.
19) The expression vector according to any of embodiments 11-18, wherein the proteolytic cleavage site is a dibasic amino acid motif; preferably the proteolytic cleavage site is a Lys-Arg motif or an Arg-Arg motif.
20) The expression vector according to any of embodiments 11-19, wherein the proteolytic cleavage site is a KexB cleavage site.
21) A fungal host cell comprising in its genome:

I) a nucleic acid construct comprising a heterologous promoter operably linked to:
a) a polynucleotide encoding a signal peptide; and
b) at least two polynucleotides encoding one or more polypeptide of interest;
wherein the at least two polynucleotides encoding one or more polypeptide of interest are each separated by a linker polynucleotide encoding a linker polypeptide comprising a proteolytic cleavage site; and
wherein the signal peptide, the one or more polypeptide of interest and the linker polypeptide(s) comprising a proteolytic cleavage site are encoded in frame as a single polypeptide;

and/or

II) an expression vector comprising said nucleic acid construct.

22) The fungal host cell according to claim 21, wherein the at least two polynucleotides encode at least two polypeptides of interest having the same amino acid sequence, and wherein the at least two polypeptides of interest are secreted as separate polypeptides having the same amino acid sequence.
23) The fungal host cell according to any of embodiments 21-22, said fungal host cell being a yeast host cell; preferably the yeast host cell is selected from the group consisting of Candida, Hansenula, Kluyveromyces, Pichia, Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
24) The fungal host cell according to any of embodiments 21-22, said fungal host cell being a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus niger, Aspergillus oryzae, Fusarium venenatum, and Trichoderma reesei; most preferably the filamentous fungal host cell is an Aspergillus oryzae cell.
25) The fungal host cell according to any of embodiments 21-24, wherein the signal peptide has a sequence identity of at least 80% to SEQ ID NO: 2; preferably the signal peptide comprises or consists of SEQ ID NO: 2.
26) The fungal host cell according to any of embodiments 21-25, wherein the polynucleotide encoding the signal peptide has a sequence identity of at least 80% to SEQ ID NO: 1; preferably the polynucleotide encoding the signal peptide comprises or consists of SEQ ID NO: 1.
27) The fungal host cell according to any of embodiments 21-26, wherein the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the polypeptide of interest is a lipase.
28) The fungal host cell according to any of embodiments 21-27, wherein the one or more polypeptide of interest has a sequence identity of at least 80% to the mature polypeptide of SEQ ID NO: 5; preferably the one or more polypeptide of interest comprises or consists of the mature polypeptide of SEQ ID NO: 5.
29) The fungal host cell according to any of embodiments 21-28, wherein the at least two polynucleotides encoding one or more polypeptide of interest have a sequence identity of, independently, at least 80% to the mature polypeptide coding sequence of SEQ ID NO: 4; preferably the at least two polynucleotides encoding one or more polypeptide of interest comprise or consist of the mature polypeptide coding sequence of SEQ ID NO: 4.
30) The fungal host cell according to any of embodiments 21-29, wherein the linker polypeptide comprises at least 10 amino acids.
31) The fungal host cell according to any of embodiments 21-30, wherein the proteolytic cleavage site is a dibasic amino acid motif; preferably the proteolytic cleavage site is a Lys-Arg motif or an Arg-Arg motif.
32) The fungal host cell according to any of embodiments 21-31, wherein the proteolytic cleavage site is a KexB cleavage site.
33) The fungal host cell according to any of embodiments 21-32, wherein proteolytic cleavage takes place intracellularly OR wherein proteolytic cleavage takes place extracellularly during and/or after cultivation of the fungal host cell.
34) A method of producing one or more polypeptide of interest, said method comprising:

A) providing a fungal host cell comprising:
I) a nucleic acid construct comprising a heterologous promoter operably linked to:
a) a polynucleotide encoding a signal peptide; and
b) at least two polynucleotides encoding one or more polypeptide of interest;
wherein the at least two polynucleotides encoding one or more polypeptide of interest are each separated by a linker polynucleotide encoding a linker polypeptide comprising a proteolytic cleavage site; and
wherein the signal peptide, the one or more polypeptide of interest and the linker polypeptide(s) comprising a proteolytic cleavage site are encoded in frame as a single polypeptide;
and/or
II) an expression vector comprising said nucleic acid construct.

B) cultivating said host cell under conditions conducive for expression of the one or more polypeptide of interest; and optionally

C) recovering the one or more polypeptide of interest.

35) The method according to claim 34, wherein the at least two polynucleotides encode at least two polypeptides of interest having the same amino acid sequence, and wherein the at least two polypeptides of interest are secreted as separate polypeptides having the same amino acid sequence.
36) The method according to any of embodiments 34-35, wherein the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the one or more polypeptide of interest is a lipase.
37) The method according to any of embodiments 34-36, wherein the fungal host cell is a yeast host cell; preferably the yeast host cell is selected from the group consisting of Candida, Hansenula, Kluyveromyces, Pichia, Saccharomyces, Schizosaccharomyces, and Yarrowia cell; more preferably the yeast host cell is selected from the group consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, and Yarrowia lipolytica cell.
38) The method according to any of embodiments 34-36, wherein fungal host cell is a filamentous fungal host cell; preferably the filamentous fungal host cell is selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma cell; more preferably the filamentous fungal host cell is selected from the group consisting of Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride cell; even more preferably the filamentous host cell is selected from the group consisting of Aspergillus niger, Aspergillus oryzae, Fusarium venenatum, and Trichoderma reesei; most preferably the filamentous fungal host cell is an Aspergillus oryzae cell.
39) The method according to any of embodiments 34-38, wherein the signal peptide has a sequence identity of at least 80% to SEQ ID NO: 2; preferably the signal peptide comprises or consists of SEQ ID NO: 2.
40) The method according to any of embodiments 34-39, wherein the polynucleotide encoding the signal peptide has a sequence identity of at least 80% to SEQ ID NO: 1; preferably the polynucleotide encoding the signal peptide comprises or consists of SEQ ID NO: 1.
41) The method according to any of embodiments 34-40, wherein the one or more polypeptide of interest comprises an enzyme; preferably the enzyme is selected from the group consisting of hydrolase, isomerase, ligase, lyase, oxidoreductase, or transferase; more preferably an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, nuclease, oxidase, pectinolytic enzyme, peroxidase, phosphodiesterase, phytase, polyphenoloxidase, proteolytic enzyme, ribonuclease, transglutaminase, xylanase, and beta-xylosidase; most preferably the one or more polypeptide of interest is a lipase. 42) The method according to any of embodiments 34-41, wherein the one or more polypeptide of interest has a sequence identity of at least 80% to the mature polypeptide of SEQ ID NO: 5; preferably the one or more polypeptide of interest comprises or consists of the mature polypeptide of SEQ ID NO: 5.
43) The method according to any of embodiments 34-42, wherein the at least two polynucleotides encoding one or more polypeptide of interest have a sequence identity of, independently, at least 80% to the mature polypeptide coding sequence of SEQ ID NO: 4; preferably the at least two polynucleotides encoding one or more polypeptide of interest comprise or consist of the mature polypeptide coding sequence of SEQ ID NO: 4.
44) The method according to any of embodiments 34-43, wherein the linker polypeptide comprises at least 10 amino acids.
45) The method according to any of embodiments 34-44, wherein the proteolytic cleavage site is a dibasic amino acid motif; preferably the proteolytic cleavage site is a Lys-Arg motif or an Arg-Arg motif.
46) The method according to any of embodiments 34-45, wherein the proteolytic cleavage site is a KexB cleavage site.
47) The method according to any of embodiments 34-46, wherein proteolytic cleavage takes place intracellularly OR wherein proteolytic cleavage takes place extracellularly during and/or after cultivation of the fungal host cell.

The present invention is further described by the following Examples that should not be construed as limiting the scope of the invention.

### Examples

### Materials and Methods

General methods of PCR, cloning, ligation, nucleotides etc. are well-known to a person skilled in the art and may for example be found in ‘Molecular Cloning: A Laboratory Manual’, Sambrook et al. (1988), Cold Spring Harbor Lab., Cold Spring Harbor, N.Y.; Ausubel, F. M. et al. (eds.); ‘Current Protocols in Molecular Biology’, John Wiley and Sons (1995); Harwood, C. R., and Cutting, S. M. (eds.); DNA Cloning: A Practical Approach, Volumes I and II′, D. N. Glover ed. (1985); ‘Oligonucleotide Synthesis’, M. J. Gait ed. (1984); ‘Nucleic Acid Hybridization’, B. D. Hames & S. J. Higgins eds (1985); ‘A Practical Guide To Molecular Cloning’, B. Perbal (1984).

Chemicals used as buffers and substrates were commercial products of at least rea-gent grade.

### Aspergillus Transformation

Aspergillus transformation was done as described in U.S. Pat. No. 9,487,767. Transformants that had repaired the target niaD-gene and contained the pyrG gene were selected for its ability to grow on minimal plates containing nitrate as nitrogen source and thiamine (Cove D. J., 1966. Biochem. Biophys. Acta 113:51-56). After 5-7 days of growth at 30 degrees C., stable transformants appeared as vigorously growing and sporulating colonies. Transformants were purified through conidiation.

### Strain Cultivation

The transformed cells are cultivated in a nutrient medium suitable for production of the lipase protein using methods well known in the art. For example, the cells may be cultivated by shake flask cultivation (in which 10 mL YPD medium (2 g/L yeast extract, 2 g/L peptone and 2% glucose) were inoculated with spores from a transformant and incubated at 30 degrees C. for 4 days), and small-scale or large-scale fermentation (including e.g., batch or fed-batch fermentation) in laboratory or industrial fermentors performed in a suitable medium and under conditions allowing the lipase protein to be expressed and recovered. The cultivation takes place in a suitable nutrient medium comprising carbon and nitrogen sources and inorganic salts, using procedures known in the art. Suitable media are available from commercial suppliers or may be prepared according to published compositions. The lipase protein is secreted into the nutrient medium and can be recovered directly here from.

### In-Fusion Cloning

In-Fusion Cloning was done using the In-Fusion cloning kit and manuals supplied by Clontech Laboratories, Inc.

Copy Number Determination by ddPCR

Copy number determination was performed by ddPCR using BioRad QX200™ Droplet Generator and QX200™ Droplet Reader using Biorad QuantaSoft™ version 1.7.4.0917, according to the manufacturer, using oliC as single copy gene.

### SDS-PAGE

Protein production was visualized by SDS-PAGE analysis using Criterion™ XT precast gels, 10% Bis-Tris, from BIO-RAD and was run and stained with Coomassie blue as recommended by the manufacturer.

### Plasmids

pAT652 is described in Example 1.

pAT1509 is described in Example 1.

### Strain

The A. oryzae host strain is derived from BECh2 which is described in WO 2000/39322.

### Example 1. Increased Production of Lipase in Aspergillus oryzae Using an Expression Cassette Containing Two Lipase Molecules in One mRNA (Tandem Construct)

The purpose of this experiment was to construct a plasmid for expression of the lipase lipVR originally isolated from Valsaria rubricosa (WO 2018/150021, mature polypeptide of SEQ ID NO: 5) as tandem construct in an A. oryzae strain, i.e., containing two lipase molecules in a single polypeptide (FIG. 1). The rationale for this is that if signal peptide removal and translocation of newly synthesized polypeptides into the endoplasmic reticulum (ER) is a rate limiting step for protein secretion in fungi, coupling one signal peptide to two or more polypeptides of interest should aid in increasing secretion.

1A. Construction of pAT652 and pAT1509 for Expression of lipVR as Singlet or Tandem

The purpose of this experiment was to construct a plasmid for expression of lipVR (WO 2018/150021) in an A. oryzae strain.

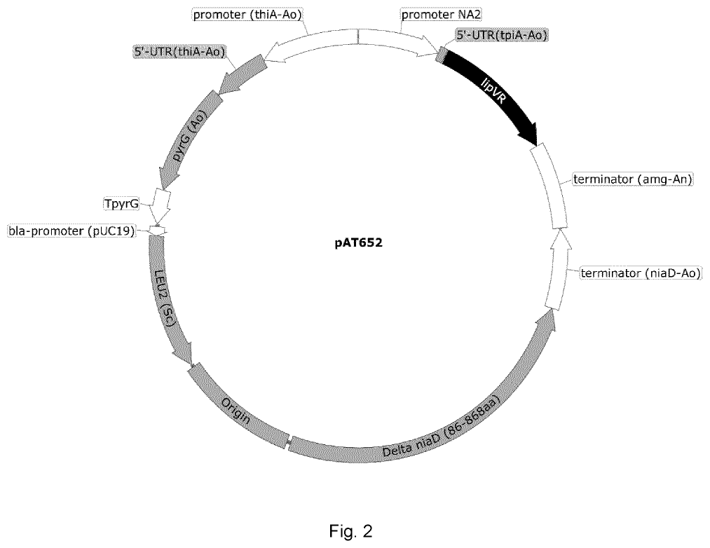

Plasmid pAT652 (FIG. 2) containing the native sequence of lipVR (including its native signal sequence) was constructed by using the In-Fusion Cloning® HD EcoDry™ cloning kit to clone an amplified lipVR gene (SEQ ID NO: 3) using primers lipVR-A (SEQ ID NO: 7) and lipVR-B (SEQ ID NO: 8) into a NotI+AsiSI-digested plasmid derived from pCOIs1175 (WO 2013/178674).

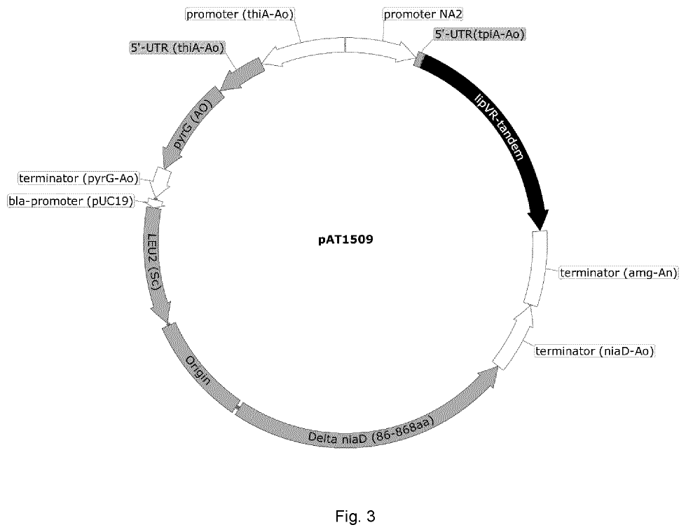

Plasmid pAT1509, containing downstream of the Pna2 promoter sequence, the following elements 1) the native lipVR-gene lacking the stop-codon (where the native signal peptide sequence was replaced with the signal peptide sequence of Cutinase pre-pro-sequence (CPP)), followed by a KexB-cleavage sequence (coding for amino acids Lys-Arg), a new DNA sequence coding for the Cutinase Pro sequence devoid of the pre-region and finally followed by a second, codon-optimised, lipVR gene (optimized for A. oryzae and lacking introns) (FIG. 3). The lipVR-tandem expression cassette (SEQ ID NO: 9) was constructed by Splicing by Overlap Extension amplification (Higuchi et al, 1988; Horton et al., 2013) using primers lipVR-C (SEQ ID NO: 10) and lipVR-D (SEQ ID NO: 11) into a NotI+AsiSI-digested plasmid derived from pCOIs1175 (WO 2013/178674).

1B. Construction and Analysis of A. oryzae Strains Expressing lipVR as Singlet or Tandem in Multiple Copies

Plasmids pAT652 (singlet) and pAT1509 (tandem) were individually used for transformation of A. oryzae host strains, and transformants were selected for its ability to grow on sucrose medium supplemented with sodium nitrate (as nitrogen source) and thiamine (down-regulation expression of pyrG-marker for increased copy number preference), but lacking uridine (selection for pyrG complementation).

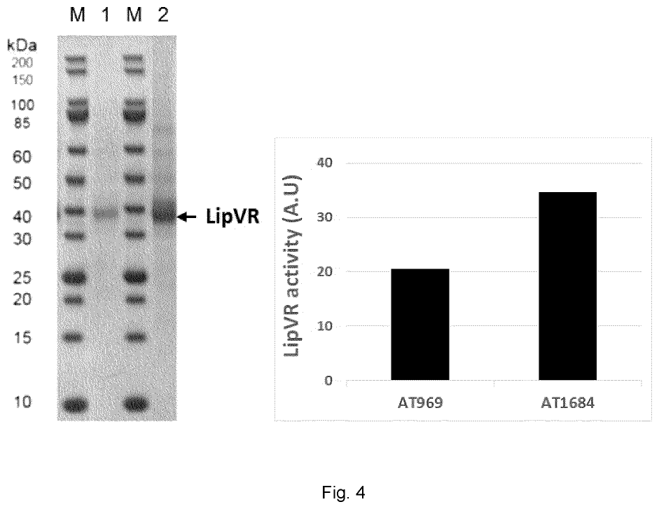

Selected transformants were cultivated in 10 mL YPD for 4 days at 30° C. and supernatants were analysed for lipase production by SDS-PAGE. Identified transformants producing lipase were genetically characterized for the copy number of the introduced expression construct and selected for fermentation. Strain AT969 contains 18 copies of the singlet lipVR cassette inserted into the genome (or 1 SP per LipVR unit), while strain AT1684 contains 9 copies of the tandem lipVR cassette, thereby 18 LipVR “units” made as 9 tandem molecules with a single SP (or 1 SP per 2 LipVR units). Samples collected from fermentation at day 7 were analyzed for lipase production by SDS-PAGE and lipolytic activity assays (FIG. 4). As shown, a significant increase of secreted LipVR in the tandem strain AT1684 compared to the singlet strain AT969 (75% increased lipolytic activity, FIG. 4).

These results strongly suggest that the use of a tandem lipVR expression cassette dramatically increases the yield of secreted and functionally active enzyme under strong expression conditions at relevant high copy numbers in a production strain background.

### Example 2. Increased Enzyme Production and Conditions for Correct Processing of the Enzyme Units

The purpose of this experiment was to investigate whether A. oryzae strains like AT1684 constructed using multiple copies of a tandem expression cassette and a suitable promoter such as the Pna2 promoter (WO 2012/160093) can be used not only to increase enzyme yields (see Example 1), but also to ensure correct processing of the given polypeptide of interest, thus enabling the use of this approach within industrial enzyme production where it is important that the produced enzyme molecules are functional and identical in order to ensure uniform activity.

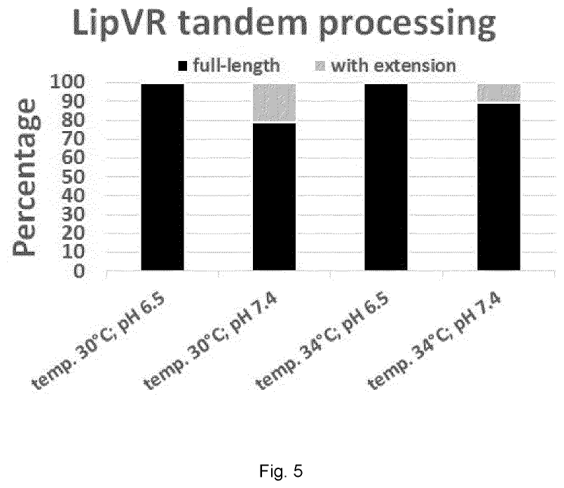

LipVR is a 30 kDa protein that runs at the apparent molecular weight of 40 kDa. Fermentation of AT1684 was performed at two different temperatures (30 and 34° C.) and two different pH values (6.5 and 7.4). MS analysis was performed on the culture supernatants to investigate the composition of LipVR derived molecules. As shown in FIG. 5, a high level of correct processing of LipVR occurs in all conditions tested after 7 days of cultivation. Remarkably, complete processing of LipVR was obtained at pH 6.5 regardless of the growth temperature, providing evidence that these growth conditions are sufficient for the processing of a tandem protein without, e.g., the requirement for overexpression of KexB. The exact conditions (temperature, pH, etc.) required for complete processing by KexB will depend on the fungal host cell of choice. The skilled person will be able to optimize the conditions for any given fungal host cell of choice using methods known in the art.

### CONCLUSION

When comparing A. oryzae strains containing identical copy numbers of the same polypeptide of interest configured as either one signal peptide-one polypeptide of interest (singlet) or one signal peptide-two polypeptide of interest (tandem), a two-fold increase in polypeptide yield was observed with the tandem configuration. Moreover, using nucleic acid constructs of the present invention leads to correct and uniform processing of each copy of the polypeptide of interest at different growth conditions. These results provide unequivocal evidence that remarkable yield increase of industrial functional enzymes can be obtained using genetic constructions containing one SP and more than one CDS of the enzyme gene of interest separated by protease cleavage recognition sites. Additionally, we demonstrate that correct and complete processing of the polypeptide occurs leading to the production of a single and functional LipVR molecule.

### REFERENCES

Aviram N, Schuldiner M (2017). Targeting and translocation of proteins to the endoplasmic reticulum at a glance. J Cell Sci 130: 4079-4085.
Higuchi R, Krummel B, Saiki R (1988). A general method of in vitro preparation and specific mutagenesis of DNA fragments: study of protein and DNA interactions. Nucleic Acids Res. 16 (15): 7351-7367.
Horton R M, Cai Z, Ho S N, and Pease L R (2013). Gene Splicing by Overlap Extension: Tailor-Made Genes Using the Polymerase Chain Reaction. BioTechniques 54 (3): 129-133.
Voss M, Schröder B, Fluhrer R (2013). Mechanism, specificity and physiology of signal peptide peptidase (SPP) and SPP-like proteases. Bioch Biophys Acta 1828: 2828-2839.

1-16. (canceled)

17. A nucleic acid construct comprising a heterologous promoter operably linked to:
a) a polynucleotide encoding a signal peptide; and
b) at least two polynucleotides encoding one or more polypeptides of interest;
wherein the at least two polynucleotides encoding one or more polypeptide of interest are each separated by a linker polynucleotide encoding a linker polypeptide comprising a proteolytic cleavage site;
wherein the signal peptide, the one or more polypeptides of interest and the linker polypeptide(s) comprising a proteolytic cleavage site are encoded in frame as a single polypeptide, and
wherein the at least two polynucleotides encode at least two polypeptides of interest having the same amino acid sequence, and wherein the at least two polypeptides of interest are secreted as separate polypeptides having the same amino acid sequence.

18. The nucleic acid construct according to claim 17, wherein the signal peptide has an amino acid sequence having at least 80% sequence identity to SEQ ID NO: 2.

19. The nucleic acid construct according to claim 17, wherein the signal peptide has an amino acid sequence that comprises or consists of SEQ ID NO: 2.

20. The nucleic acid construct according to claim 17, wherein the polynucleotide encoding the signal peptide has a sequence of at least 80% sequence identity to SEQ ID NO: 1.

21. The nucleic acid construct according to claim 17, wherein the polynucleotide encoding the signal peptide has a sequence that comprises or consists of SEQ ID NO: 1.

22. The nucleic acid construct according to claim 17, wherein the one or more polypeptide of interest comprises an enzyme.

23. The nucleic acid construct according to claim 22, wherein the enzyme is a lipase.

24. The nucleic acid construct according to claim 17, wherein the one or more polypeptides of interest has an amino acid sequence having at least 80% sequence identity to the mature polypeptide of SEQ ID NO: 5.

25. The nucleic acid construct according to claim 17, wherein the one or more polypeptides of interest has an amino acid sequence that comprises or consists of the mature polypeptide of SEQ ID NO: 5.

26. The nucleic acid construct according to claim 17, wherein the at least two polynucleotides encoding one or more polypeptides of interest, independently, have a sequence having at least 80% sequence identity to the mature polypeptide coding sequence of SEQ ID NO: 4.

27. The nucleic acid construct according to claim 17, wherein the at least two polynucleotides encoding one or more polypeptide of interest, independently, have a sequence that comprises or consists of the mature polypeptide coding sequence of SEQ ID NO: 4.

28. The nucleic acid construct according to claim 17, wherein the linker polypeptide comprises at least 10 amino acids.

29. The nucleic acid construct according to claim 17, wherein the proteolytic cleavage site is a dibasic amino acid motif.

30. The nucleic acid construct according to claim 17, wherein the proteolytic cleavage site is a Lys-Arg motif or an Arg-Arg motif.

31. The nucleic acid construct according to claim 17, wherein the proteolytic cleavage site is a KexB cleavage site.

32. An expression vector comprising a nucleic acid construct according to claim 17.

33. A fungal host cell comprising a nucleic acid construct according to claim 17.

34. The fungal host cell according to claim 33, when the cell is a yeast host cell.

35. The fungal host cell according to claim 33, wherein the cell is a filamentous fungal host cell.

36. The fungal host cell according to claim 33, wherein the cell is an Aspergillus oryzae cell.

37. A method of producing one or more polypeptide of interest, said method comprising:
a) providing a fungal host cell according to claim 33;
b) cultivating said host cell under conditions conducive for expression of the one or more polypeptides of interest; and optionally
c) recovering the one or more polypeptides of interest.

38. The method according to claim 37, wherein the one or more polypeptides of interest is an enzyme.

39. The method according to claim 37, wherein the one or more polypeptides of interest is a lipase.


SEQUENCE LISTING (11 sequences)

SEQ ID NO: 1 - DNA, 105 bp, Artificial Sequence
  DNA sequence of CPP used as signal peptide
atgaagttcttcaccaccatcctcagcaccgccagccttgttgctgctctccccgccgctgttgactcgaaccataccccggccgctcctgaacttgttgcccgg

SEQ ID NO: 2 - protein, 35 aa, Artificial Sequence
  Amino acid sequence of CPP used as signal peptide
MKFFTTILSTASLVAALPAAVDSNHTPAAPELVAR

SEQ ID NO: 3 - DNA, 1059 bp, Valsaria rubricosa
atgaagtccgcttcgatcttactcagggtagctgccctcctcctccctgctgtatctgcactgccacttgaaagaagaggtatggacgaactatcctagcgatcagtgtgtctattttgcctaacctagcaaagctatatccgcggatctcctggcaaccttcagcctcttcgagcagttcgcagccgcagcatattgtccggataacaacgacagtcccgacaccaagcttacttgctctgtcggaaactgcccgcttgtcgaagctgacacgaccagcacggtcactgaattcgaaaagtacatcttacacgaccccgttcacctacagacaaagtcccagctaacgtccacctctatctctgtccctttagctcgctcgaaaccgacgtcactggctacgtcgcgactgacagcacacgagagctcatcgttgtggcattccgcgggagttcctcgatccggaactggatcgccgacatcgactttcccttcaccgacaccgacctctgcgatggctgccaggcagcctcgggcttctggacgtcctggacggaggcacggacaggggtgctggcggcggtggcgagcgctgccgcggccaacccgtcctataccgttgccgtgacgggccacagcctcggcggggccgtggccgcgctggccgctggcgccctccggaacgcgggctacacggtcgcgctatacagcttcggagcgcctcgcgtgggtgacgagaccctcagcgagtacatcactgcgcaggcgggtggaaactaccgcatcacgcacctcaacgacccagtgccgaagctgcccccgctgctcctggggtatcgccacatcagcccggaatactacatcagcagcgggaacaacgtgaccgtgacggcggatgacgtggaggagtacaccggcacgatcaacctgagtgggaacacgggcgatctgacgttcgacacggatgcgcacagttggtacttcaacgagatcggggcatgcgatgatggtgaggctttggagtggaagaagcggggggtagaagttcagtgggtttaa

SEQ ID NO: 4 - DNA, 930 bp, Artificial Sequence
  cDNA of DNA sequence of LipVR gene
  CDS (1)..(927)
  sig_peptide (1)..(60)
  mat_peptide (61)..(927)
atgaagtccgcttcgatcttactcagggtagctgccctcctcctccctgctgtatctgcactgccacttgaaagaagagctatatccgcggatctcctggcaaccttcagcctcttcgagcagttcgcagccgcagcatattgtccggataacaacgacagtcccgacaccaagcttacttgctctgtcggaaactgcccgcttgtcgaagctgacacgaccagcacggtcactgaattcgaaaactcgctcgaaaccgacgtcactggctacgtcgcgactgacagcacacgagagctcatcgttgtggcattccgcgggagttcctcgatccggaactggatcgccgacatcgactttcccttcaccgacaccgacctctgcgatggctgccaggcagcctcgggcttctggacgtcctggacggaggcacggacaggggtgctggcggcggtggcgagcgctgccgcggccaacccgtcctataccgttgccgtgacgggccacagcctcggcggggccgtggccgcgctggccgctggcgccctccggaacgcgggctacacggtcgcgctatacagcttcggagcgcctcgcgtgggtgacgagaccctcagcgagtacatcactgcgcaggcgggtggaaactaccgcatcacgcacctcaacgacccagtgccgaagctgcccccgctgctcctggggtatcgccacatcagcccggaatactacatcagcagcgggaacaacgtgaccgtgacggcggatgacgtggaggagtacaccggcacgatcaacctgagtgggaacacgggcgatctgacgttcgacacggatgcgcacagttggtacttcaacgagatcggggcatgcgatgatggtgaggctttggagtggaagaagcggggggtagaagttcagtgggtttaa

SEQ ID NO: 5 - protein, 309 aa, Artificial Sequence
  Synthetic Construct
MKSASILLRVAALLLPAVSALPLERRAISADLLATFSLFEQFAAAAYCPDNNDSPDTKLTCSVGNCPLVEADTTSTVTEFENSLETDVTGYVATDSTRELIVVAFRGSSSIRNWIADIDFPFTDTDLCDGCQAASGFWTSWTEARTGVLAAVASAAAANPSYTVAVTGHSLGGAVAALAAGALRNAGYTVALYSFGAPRVGDETLSEYITAQAGGNYRITHLNDPVPKLPPLLLGYRHISPEYYISSGNNVTVTADDVEEYTGTINLSGNTGDLTFDTDAHSWYFNEIGACDDGEALEWKKRGVEVQWV

SEQ ID NO: 6 - protein, 289 aa, Valsaria rubricosa
LPLERRAISADLLATFSLFEQFAAAAYCPDNNDSPDTKLTCSVGNCPLVEADTTSTVTEFENSLETDVTGYVATDSTRELIVVAFRGSSSIRNWIADIDFPFTDTDLCDGCQAASGFWTSWTEARTGVLAAVASAAAANPSYTVAVTGHSLGGAVAALAAGALRNAGYTVALYSFGAPRVGDETLSEYITAQAGGNYRITHLNDPVPKLPPLLLGYRHISPEYYISSGNNVTVTADDVEEYTGTINLSGNTGDLTFDTDAHSWYFNEIGACDDGEALEWKKRGVEVQWV

SEQ ID NO: 7 - DNA, 41 bp, Artificial Sequence
  Primer lipVR-A
tacacaactgggggccaccatgaagtccgcttcgatcttac

SEQ ID NO: 8 - DNA, 38 bp, Artificial Sequence
  Primer lipVR-B
gtgtcagtcaccgcgttaaacccactgaacttctaccc

SEQ ID NO: 9 - DNA, 2028 bp, Artificial Sequence
  DNA sequence of lipVR-tandem
atgaagttcttcaccaccatcctcagcaccgccagccttgttgctgctctccccgccgctgttgactcgaaccataccccggccgctcctgaacttgttgcccggctgccacttgaaagaagaggtatggacgaactatcctagcgatcagtgtgtctattttgcctaacctagcaaagctatatccgcggatctcctggcaaccttcagcctcttcgagcagttcgcagccgcagcatattgtccggataacaacgacagtcccgacaccaagcttacttgctctgtcggaaactgcccgcttgtcgaagctgacacgaccagcacggtcactgaattcgaaaagtacatcttacacgaccccgttcacctacagacaaagtcccagctaacgtccacctctatctctgtccctttagctcgctcgaaaccgacgtcactggctacgtcgcgactgacagcacacgagagctcatcgttgtggcattccgcgggagttcctcgatccggaactggatcgccgacatcgactttcccttcaccgacaccgacctctgcgatggctgccaggcagcctcgggcttctggacgtcctggacggaggcacggacaggggtgctggcggcggtggcgagcgctgccgcggccaacccgtcctataccgttgccgtgacgggccacagcctcggcggggccgtggccgcgctggccgctggcgccctccggaacgcgggctacacggtcgcgctatacagcttcggagcgcctcgcgtgggtgacgagaccctcagcgagtacatcactgcgcaggcgggtggaaactaccgcatcacgcacctcaacgacccagtgccgaagctgcccccgctgctcctggggtatcgccacatcagcccggaatactacatcagcagcgggaacaacgtgaccgtgacggcggatgacgtggaggagtacaccggcacgatcaacctgagtgggaacacgggcgatctgacgttcgacacggatgcgcacagttggtacttcaacgagatcggggcatgcgatgatggtgaggctttggagtggaagaagcggggggtagaagttcagtgggttaaacgagccgctgttgactcgaaccataccccggccgctcctgaacttgttgcccggttgcctttggaaaggagggcgatttccgcagacctcctcgcgaccttctccttgttcgaacagttcgcagcagcagcctactgtcccgacaacaacgactcccctgatactaagttgacatgttcggtcggcaactgtcctttggtcgaagccgacacaacatcgaccgtgacagagttcgagaactccctcgagacagacgtcacgggttatgtggcaaccgattccacgcgtgagctcatcgtcgtcgccttcaggggatcctcgtccatccggaactggattgccgacatcgatttccccttcaccgataccgatctctgtgatggttgtcaggcagcatcgggcttctggacctcgtggacagaagcccgaacaggtgtgctcgcagccgtggcgtccgcagcagcagcgaacccctcctacacggtggcagtgacaggccattcgttgggtggtgcggtcgcagccctcgcagcaggtgccttgaggaacgcaggctacacagtggcgttgtactccttcggtgcacctcgggtcggcgacgagacattgtcggagtatatcactgcgcaggcaggtggcaactacaggattactcacctcaacgacccggtccctaagctccctccgttgctcttgggctatcgacacatttcgcctgagtattacatctcgtccggcaacaacgtcacggtgacagccgatgatgtggaggagtacactggaacaatcaacttgtcgggtaacacaggcgatttgaccttcgacacagacgcccactcgtggtatttcaacgagattggtgcgtgtgatgatggcgaggcattggaatggaagaaacgcggagtcgaagtgcagtgggtctaa

SEQ ID NO: 10 - DNA, 41 bp, Artificial Sequence
  Primer lipVR-C
tacacaactgggggccaccatgaagttcttcaccaccatcc

SEQ ID NO: 11 - DNA, 36 bp, Artificial Sequence
  Primer lipVR-D
gtgtcagtcaccgcgttagacccactgcacttcgac